(19) United States
(12) Patent Application Publication
(10) Pub. No.: US 2013/0224761 A1
(43) Pub. Date: Aug. 29, 2013

(54) NON COVALENT MOLECULAR STRUCTURE, COMPRISING A PORPHYRIN BASED GLYCOCONJUGATE, DEVICE COMPRISING THE SAME AND ITS USE FOR DETECTION OF LECTIN

(71) Applicants: Anne Imberty, Claix (FR); Sébastien Vidal, Villeurbanne (FR); Alexander Star, Pittsburgh, PA (US)

(72) Inventors: Anne Imberty, Claix (FR); Sébastien Vidal, Villeurbanne (FR); Alexander Star, Pittsburgh, PA (US)

(73) Assignees: CENTRE NATIONAL DE LA RECHERCHE SCIENTIFIQUE (CNRS), Paris (FR); UNIVERSITY OF PITTSBURGH-OF THE COMMONWEALTH SYSTEM OF HIGHER EDUCATION, Pittsburgh, PA (US); UNIVERSITE CLAUDE BERNARD LYON 1 (UCBL), Villeurbanne Cedex (FR)

(21) Appl. No.: 13/805,099

(22) PCT Filed: Jun. 16, 2011
(86) PCT No.: PCT/IB2011/052617

§ 371 Date: Mar. 4, 2013

(51) Int. Cl. G01N 33/569

(52) U.S. Cl. CPC G01N 33/56911
USPC 435/7.1; 435/287.1; 536/17.1

(57) ABSTRACT

The present invention relates to a non covalent molecular structure comprising a carbon nanostructure and a porphyrin based glycoconjugate (I) which is linked to the said carbon nanostructure by a non covalent link, the said glycoconjugate (I) having the formula (I): wherein M is a metal selected in the group comprising Fe, Ni, Zn, Cu, Mn, Cr or Co, B is a group which is present on at least one of the four phenyl group (C₆H₅) represented in (I), n is an integer from 1 to 3, that is to say that one to three B group(s) may be present on each phenyl group, and B is represented by a -A-C group wherein A is selected in the group comprising an oxygen atom, a sulfur atom, a NH group or a (CH₂)ₙ₁ group, n₁ being an integer from 1 to 10, C is a group of formula (II). The present invention also relates to an electronic device comprising the said non covalent molecular structure, and to the use of this device for the detection of a lectin involved in bacterial or viral infections. Thus the invention also relates to a method for detecting the presence of a lectin in a sample to be analysed.

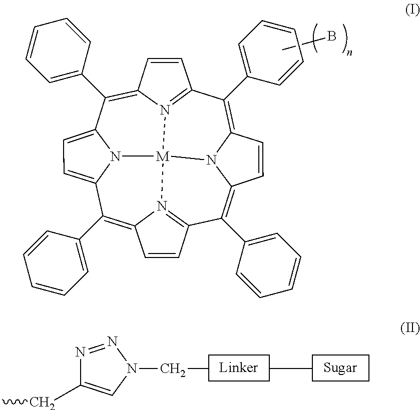

The present invention relates to novel non covalent molecular structures between carbon nanostructures and porphyrin based glycoconjugates, to a device comprising these novel molecular structures and to the use of this device for the detection of a lectin.

Lectins are proteins capable of binding to carbohydrates but devoided of any catalytic activity and they are essential to many biological processes such as cell-to-cell communication, inflammation, viral infections (HIV, influenza), cancer or bacterial adhesion. Lectins are specialized receptors which are used by several opportunistic Gram negative bacteria for specific recognition of human glycans present on tissue surface. Most lectins from opportunistic bacteria bind complex oligosaccharides such as the ones defining histo-blood group epitopes. Contrary to their counterpart in plants or animals, bacterial lectins present strong affinity towards ligands which makes them attractive targets for diagnostic.

The detection of bacterial lectins is required in the case of bacterial or viral infections and is of primary importance for public health but is also of importance in hospitals for safety purposes (most of hospital acquired infections being caused by bacteria with about 20% of these due to Pseudomonas aeruginosa) and the prevention of exposure to these agents. This is also true for outdoor environmental safety issues like the prevention of exposure to these agents through recreative waters (public swimming pools, lakes, others water reservoirs), tap waters and even for the prevention of biological terrorism.

At the present time, the detection of bacteria is classically achieved through culture-based techniques or through molecular techniques based on polymerase chain reaction (PCR). However both methods are relatively slow and not always applicable (non-culturable bacteria, impurity in DNA samples . . . ). These molecular methods can take up to a few days and require specialized skills.

An alternative to these techniques can be the use of nano-technologies for designing miniaturized and highly sensitive bioanalytical systems. The fast growing field of nanotechnology has found several applications in cell biology through quantum dots, nanofibers and carbon nanotubes.

Single-walled carbon nanotubes (SWNTs) are ideal for the design of biosensors because of their high electrical conductivity and small diameter (˜1 nm) which is comparable to individual biomolecules. Additionally, SWNTs are composed almost entirely of surface atoms allowing detection of tiny changes in the local chemical environment and thus display extreme sensitivity. These unique attributes have led researchers to incorporate SWNTs as conductive channels in solid-state electronic devices such as field-effect transistors (FETs), creating low power and ultra small electro-analytical platforms for monitoring various biomolecular interactions.

The WO 2008/044896 document relates to carbon nanotubes (CNT)-Dendron composite and a biosensor for detecting a biomolecule comprising the CNT-Dendron composite.

The WO 2009/141486 document relates to a glycolipid/carbon nanotube aggregate and to the use thereof in processes that involve interactions between carbohydrates and other biochemical species.

However none of these documents relate to the detection of lectins.

Therefore, there is a need to develop advantageous diagnostic methods permitting the detection of lectins.

One aim of the invention is to provide a method for detecting the presence of a lectin involved in bacterial or viral infections which is fast (less than 1 minute), accurate and quantitative.

Another aim of the invention is to provide a novel diagnostic method of a bacterial lectin having an excellent sensitivity.

Another aim of the invention is to provide an accurate and rapid diagnostic of the presence or not of a lectin from all bacteria, viruses and parasites that use human glycoconjugates in the early steps of infection.

In an aspect, the present invention provides a non covalent molecular structure characterized in that it comprises a carbon nanostructure and a porphyrin based glycoconjugate (I) which is linked to the said carbon nanostructure by a non covalent link, the said glycoconjugate (I) having the formula:

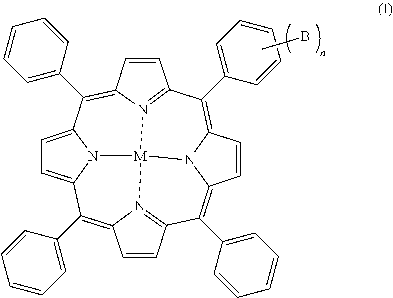

wherein
M is a metal selected in the group comprising Fe, Ni, Zn, Cu, Mn, Cr or Co,
B is a group which is present on at least one of the four phenyl group (C₆H₅) represented in (I),
n is an integer from 1 to 3, that is to say that one to three B group(s) may be present on each phenyl group,
and B is represented by a -A-C group
wherein
A is selected in the group comprising an oxygen atom (O), a sulfur atom (S), a NH group or a (CH₂)ₙ₁ group, n₁ being an integer from 1 to 10,
C is a group of formula:

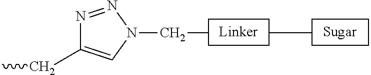

wherein
the linker is a group of formula:

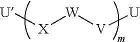

wherein
m is an integer from 0 to 15 (and preferably from 0 to 5)
U′, U=absent or is CH₂ (methylene) with the proviso that when m=0 then if one of U′ or U is absent then the other is CH₂,
X=CH₂, O, CO (carbonyl)

### W=CH₂, NH

V=CH₂, C₆H₄ (phenyl “Ph”)
the sugar is a group having at least one carbohydrate moiety and is selecting in the group comprising:

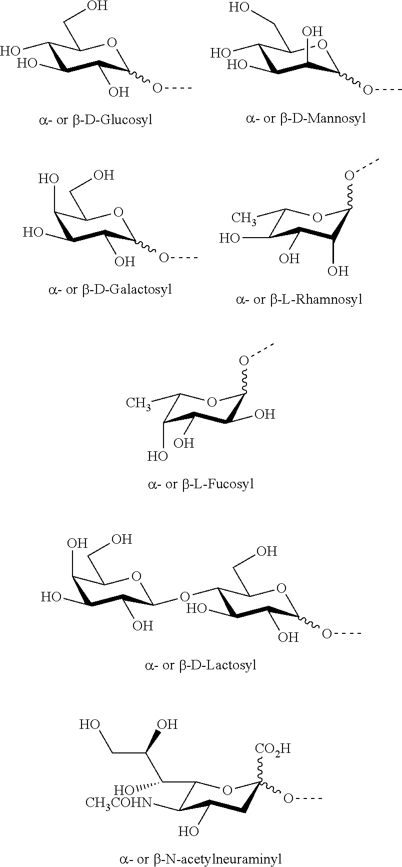

and their derivatives.

Advantageously, the above mentioned sugar derivatives in the C group are for example selected in the group comprising:

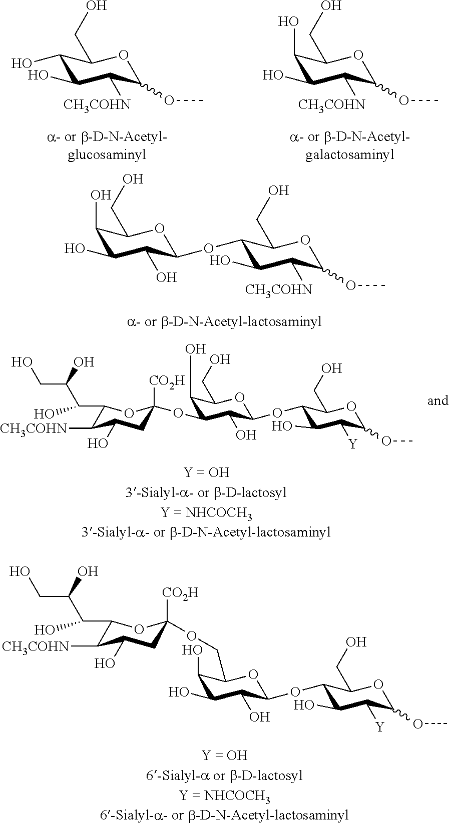

In another aspect, the above mentioned sugar derivatives in the C group are selected in the group comprising:

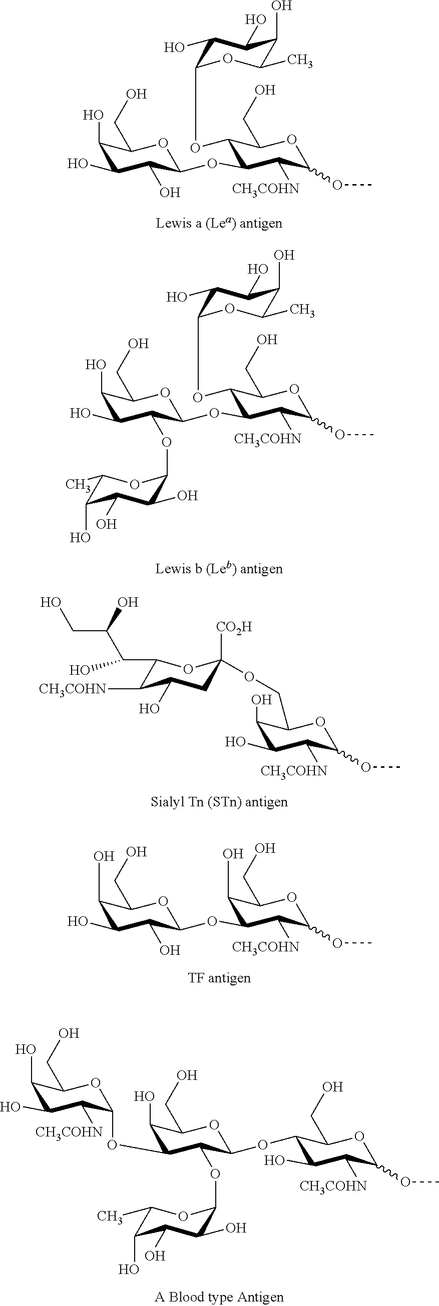

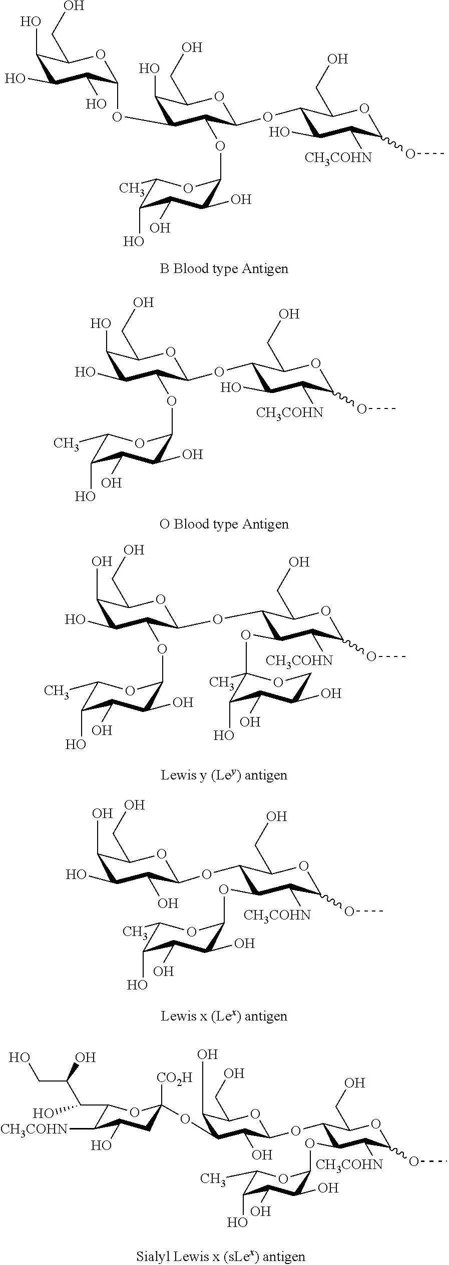

Advantageously, the linker defined in the C group of the non covalent molecular structure is selected in the group comprising:

m=0, U′=absent and U=CH₂ (i.e linker=CH₂),

m=0, U′=U=CH₂ (i.e linker=(CH₂)₂),

m=1, U′=U=absent, X=W=V=CH₂ (i.e linker=(CH₂)₃),

m=2, U′=U=absent, X=W=V=CH₂ (i.e linker=(CH₂)₆),

m=1, U′=CH₂, U=absent, X=O, W=V=CH₂ (i.e linker=CH₂—(O—CH₂—CH₂)),

m=2, U′=CH₂, U=absent, X=O, W=V=CH₂ (i.e linker=CH₂—(O—CH₂—CH₂)₂),

m=2, U′=absent, U=V=CH₂, X=CO, W=NH (i.e linker=(CO—NH—CH₂)₂—CH₂) and

m=1, U′=U=absent, X=CO, W=NH and V=Ph (i.e linker=CO—NH-Ph).

In yet another aspect of the invention, the B group of the porphyrin based glycoconjugate (I) of the non covalent molecular structure as above described is present on each of the four phenyl group and when:

n=1, B is preferably in the para-position of each phenyl group,

n=2, the two B are preferably in the two meta-position of each phenyl group,

n=3, the three B are preferably in the para-position and in the two meta-position of each phenyl group.

In a further aspect of the invention, in the porphyrin based glycoconjugate (I) of the non covalent molecular structure, A is an oxygen group, n=1 or 2 and M is Zn, the said glycoconjugate (I) being selected in the group comprising:

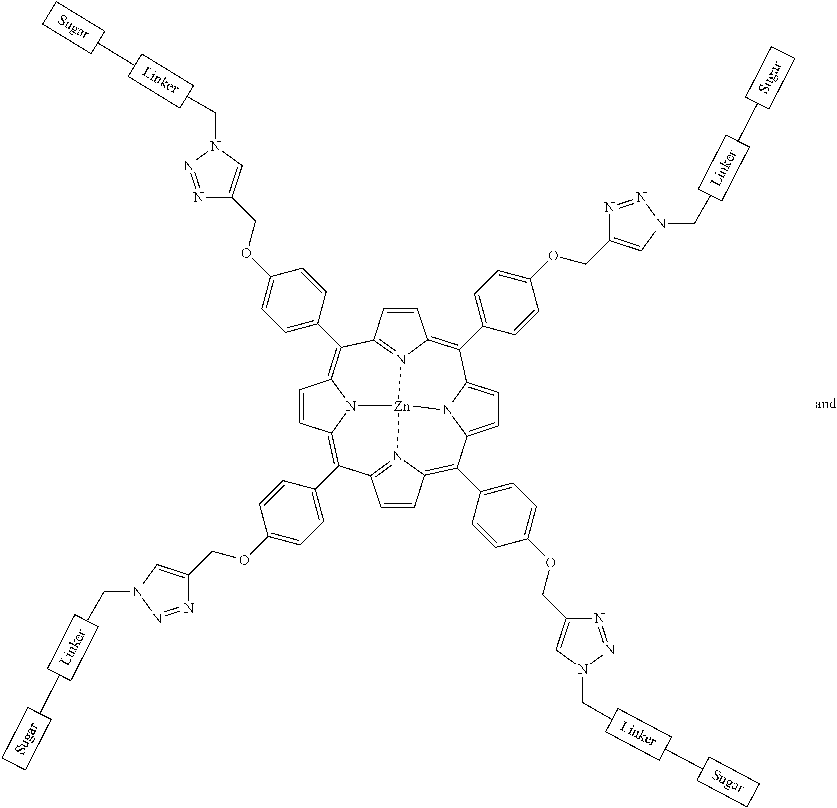

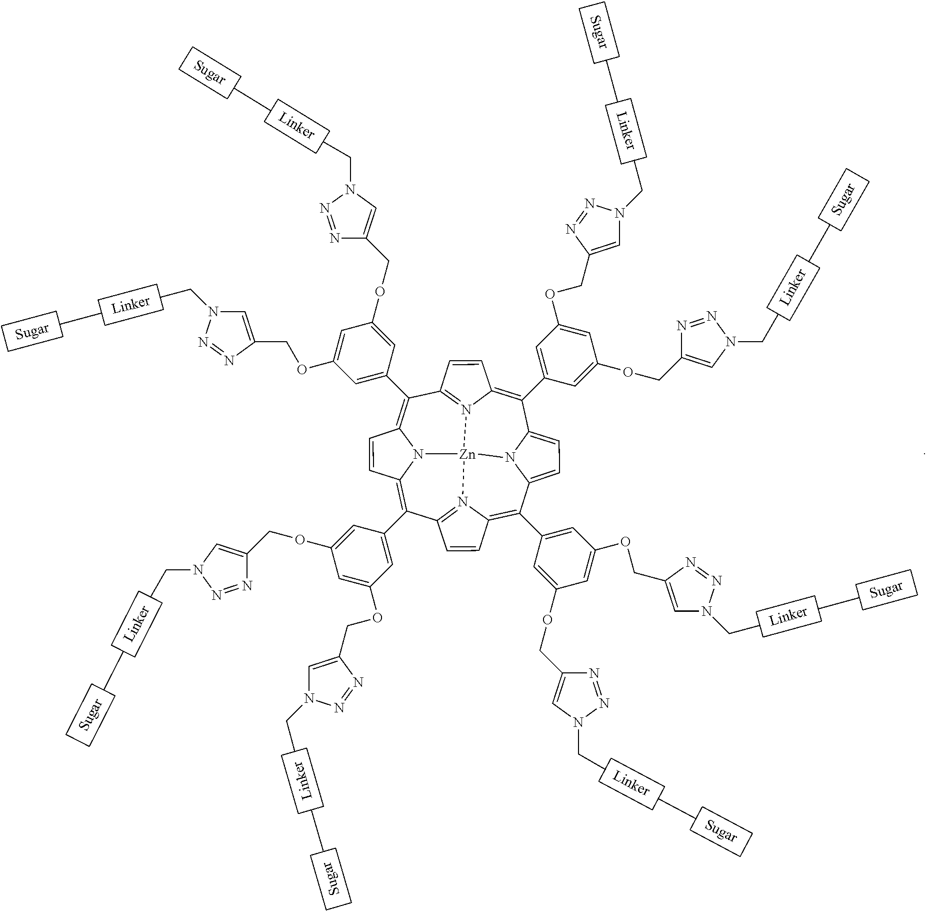

In yet a further aspect of the invention, in the porphyrin based glycoconjugate (I) of the non covalent molecular structure, the linker is CH₂—(O—CH₂—CH₂)₂ and the sugar is selected in the group comprising β-D-galactosyl, α-D-mannosyl and α-L-fucosyl.

In another aspect of the present invention, the carbon nanostructures of the non covalent molecular structure are selected in the group comprising carbon nanotubes, graphene, graphitic onions, cones, nanohorns, nanohelices, nanobarrels and fullerenes.

Advantageously, the above mentioned carbon nanostructures are preferably graphene or carbon nanotubes, the said carbon nanotubes being selected in the group comprising Single Wall Carbon Nanotubes (SWCNTs), Double Wall Carbon Nanotubes (DWCNTs), Triple Wall Carbon Nanotubes (TWCNTs) and Multi Wall Carbon Nanotubes (MWCNTs).

Graphene is a one-atom-thick planar sheet of sp²-bonded carbon atoms that are densely packed in a honeycomb crystal lattice.

In yet another aspect of the present invention, the non-covalent link between the carbon nanostructures and the glycoconjugate (I) of the non covalent molecular structure is a π-π type interaction.

The present invention also provides any device comprising a non covalent molecular structure as defined previously and capable of detecting a lectin in an aqueous solution through an electrical resistivity or conductivity.

Thus in another aspect, the present invention provides a device for detecting a lectin characterized in that it comprises a non covalent molecular structure as defined previously.

According to an aspect of the present invention, such a device could advantageously be an electronic nano-detection device comprising a field effect transistor (FET),

the said device comprising:

carbon nanostructures bridging two metal electrodes respectively called “source” (S) and “drain” (D),

a third electrode called “gate” (G) connected either to a substrate layer or to an electrode immersed in a solution covering the said device (“liquid gate”).

One of the originality of the present invention is thus the use of the said non covalent molecular structure in a device as above described for the detection of a lectin involved in bacterial or viral infections. The Inventors of the present invention have advantageously combined several knowledges of different technical fields in order to establish novel molecular structures which can be used for a diagnostic purpose (the detection of a bacterial lectin).

Thus here is used—biological knowledges about the capacity of some pathogens (bacterial lectins) to attach to human glycans (glycolipids and glycoproteins) present at the surface of human cells (that is to say the carbohydrate-lectin interactions involved in bacterial virulence)—knowledges concerning nanotechnology and the electronic devices and—hemical knowledges in order to conceive a chemical structure which will interact with the electronic device and the lectins.

In the device as described previously, the two metal electrodes (S) and (D) are spacing each other from 1 nm to 10 cm, preferably from 1 cm to 2.5 cm and more preferably from 1 μm to 10 μm.

Any metal is appropriate for preparing the electrodes (S) and (D). Examples of suitable metal can include, but are not limited to aluminium, chromium, titanium, gold and palladium.

Advantageously in the said device, the substrate layer is an insulator. Examples of suitable substrate layers can include, but are not limited to silicon dioxide layer, hafnium oxide and silicon nitrate.

According to still another aspect, the present invention also provides a method for detecting the presence of a lectin in a sample to be analysed characterized in that it comprises the following steps:

using a device as described previously,

bringing the lectin to be analysed in contact with the non covalent molecular structure as described previously,

detecting a molecular interaction between the lectin and the sugar of the porphyrin based glycoconjugate (I) of the said non covalent molecular structure, said molecular interaction being detected by a change of the conductive properties of the carbon nanostructures resulting in a change of the electric signal of the said device.

Advantageously according to the present invention, the porphyrin based glycoconjugates (I) will be used for selective attachment of targeted lectins while carbon nanostructures with their nanoscale dimensions, large surface to volume ratio and unique physical and chemical properties will aid in electronic transduction of the interaction between glycoconjugates and lectins, leading to a rapid and ultrasensitive detection.

The change in carbon nanostructures-FET conductance will be used for studying the molecular interaction between porphyrin based glycoconjugate (I) and lectin as well as to monitor the variation in lectin concentration.

The sample to be analysed can come from a pure lectin from commercial sources or isolated from recombinant production techniques, or any sample containing bacteria such as water, soils or sample of human origin.

In a general way, the method according to the present invention can be used for the detection of lectins from all bacteria, viruses and parasites that use human glycoconjugates in the early steps of infection. Advantageously, examples of suitable lectins can include, but are not limited to, those selected in the group comprising Pseudomonas aeruginosa first lectin (PA-IL), Pseudomonas aeruginosa second lectin (PA-IIL), Concanavalin A (Con A) lectin, Burkholderia cenocepacia A (Bc2L-A) lectin, Burkholderia cenocepacia B (Bc2L-B) lectin, Burkholderia cenocepacia C (Bc2L-C) lectin, Burkholderia ambifaria (Bamb541) lectin, Ralstonia solanacearum (RSL) lectin, Ralstonia solanacearum second lectin (RS-IIL) and Chromobacterium violaceum (CV-IIL) lectin.

In another aspect of the invention, the preparation of the device as above defined comprises the following steps:

forming two metal electrodes (S) and (D) on the substrate layer connected to (G),

adding, between the two electrodes (S) and (D), the carbon nanostructures and then a porphyrin based glycoconjugate (I) in order to form a non covalent molecular structure as defined.

In a further aspect of the invention, the preparation of the device as above defined comprises the following steps:

forming two metal electrodes (S) and (D) on the substrate layer connected to (G),

adding, between the two electrodes (S) and (D), a non covalent molecular structure as above defined.

In yet a further aspect of the invention, the preparation of the device as above defined comprises the following steps:

generating carbon nanostructures on the substrate layer connected to (G) (by a chemical vapour deposition (CVD) process),

forming two metal electrodes (S) and (D) around the carbon nanostructures,

adding a porphyrin based glycoconjugate (I) in order to form a non covalent molecular structure as above defined.

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention.

Reference is now made to the following examples in conjunction with the accompanying drawings.

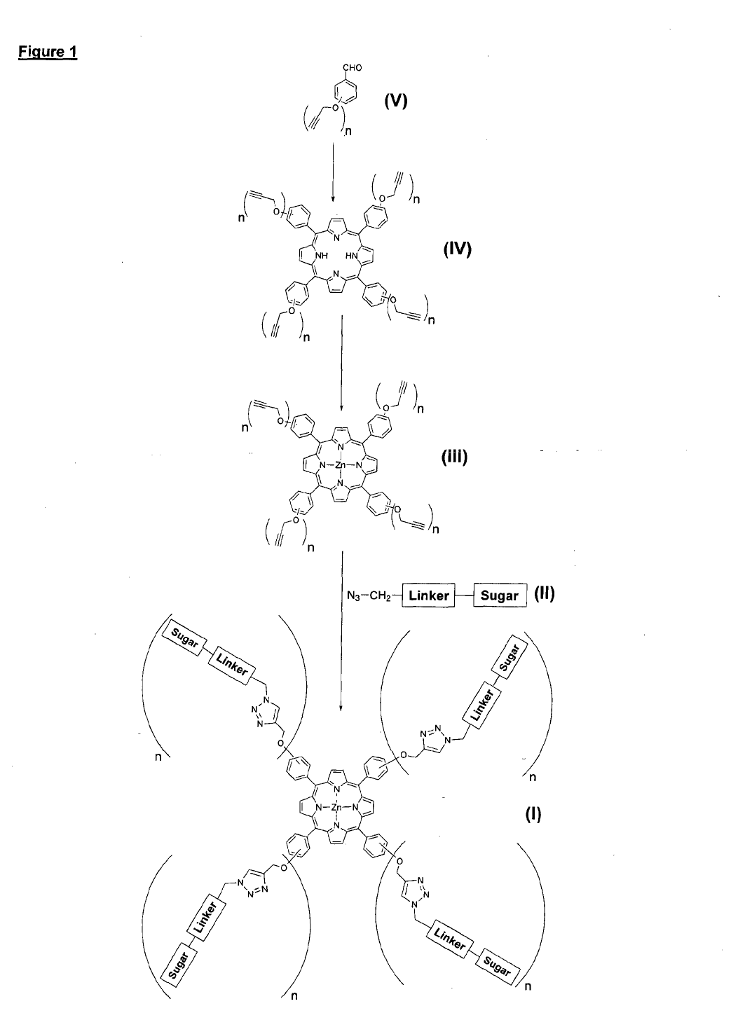

FIG. 1 is a general synthesis scheme illustrating the chemical structures and the preparation of porphyrine based glycoconjugates (I) wherein M is Zn and A is an oxygen atom.

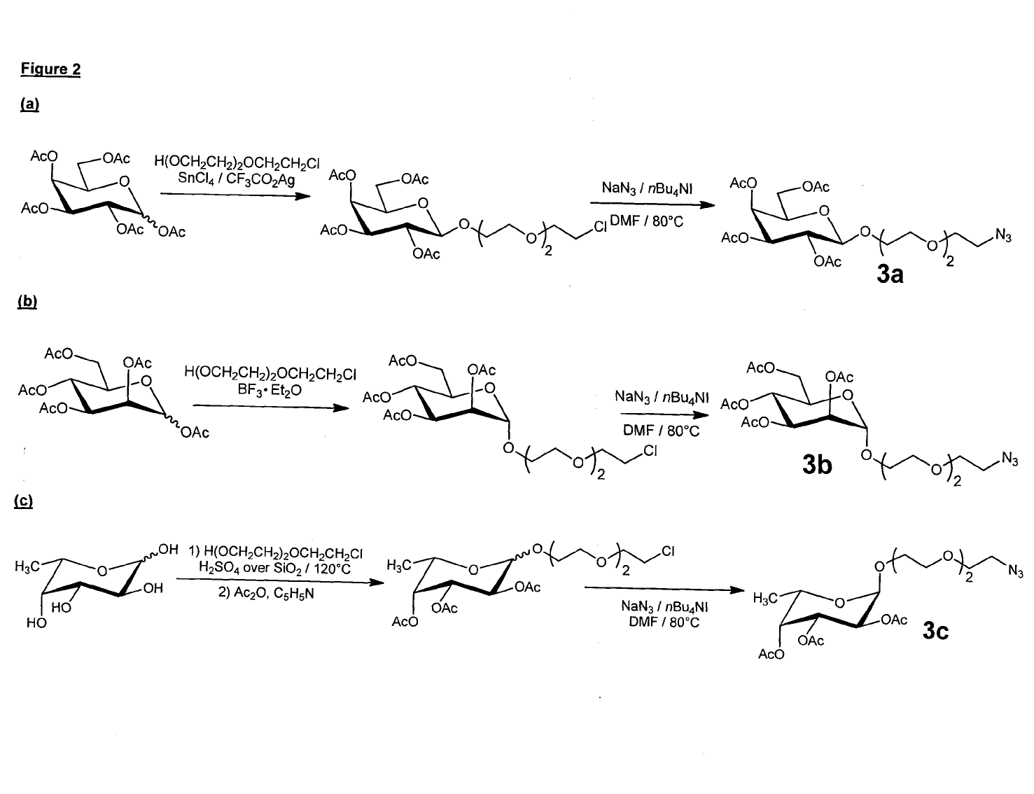

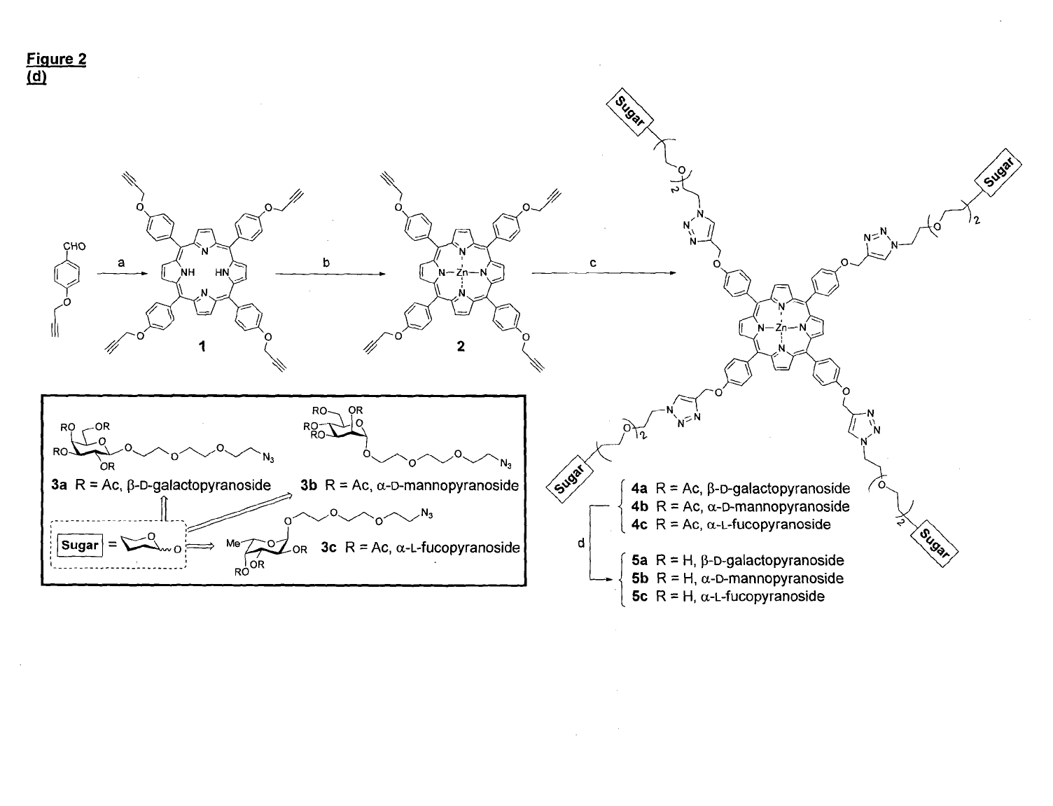

FIG. 2 represents specific synthesis schemes illustrating the general synthesis scheme of FIG. 1. More particularly FIGS. 2a, 2b and 2c represent synthesis schemes of carbohydrate azido-derivatives represented in FIG. 1 with the general formula (II) wherein Linker=CH₂—(O—CH₂—CH₂)₂ and Sugar=β-D-galactosyl (see compound named “3a”), α-D-mannosyl (see compound “3b”) and α-L-fucosyl (see compound “3c”).

FIG. 2d is a specific synthesis scheme of three porphyrin based glycoconjugates (I) wherein M=Zn, A=O, Linker=CH₂—(O—CH₂—CH₂)₂ and Sugar=β-D-galactosyl (see compound named “5a”), α-D-mannosyl (see compound “5b”) and α-L-fucosyl (see compound “5c”), n=1 and the B substituent is present on each phenyl group and is in the para position of each phenyl group.

“Ac” defined in the compounds described in FIG. 2 means “acetyl” (ie ═CH₃—CO).

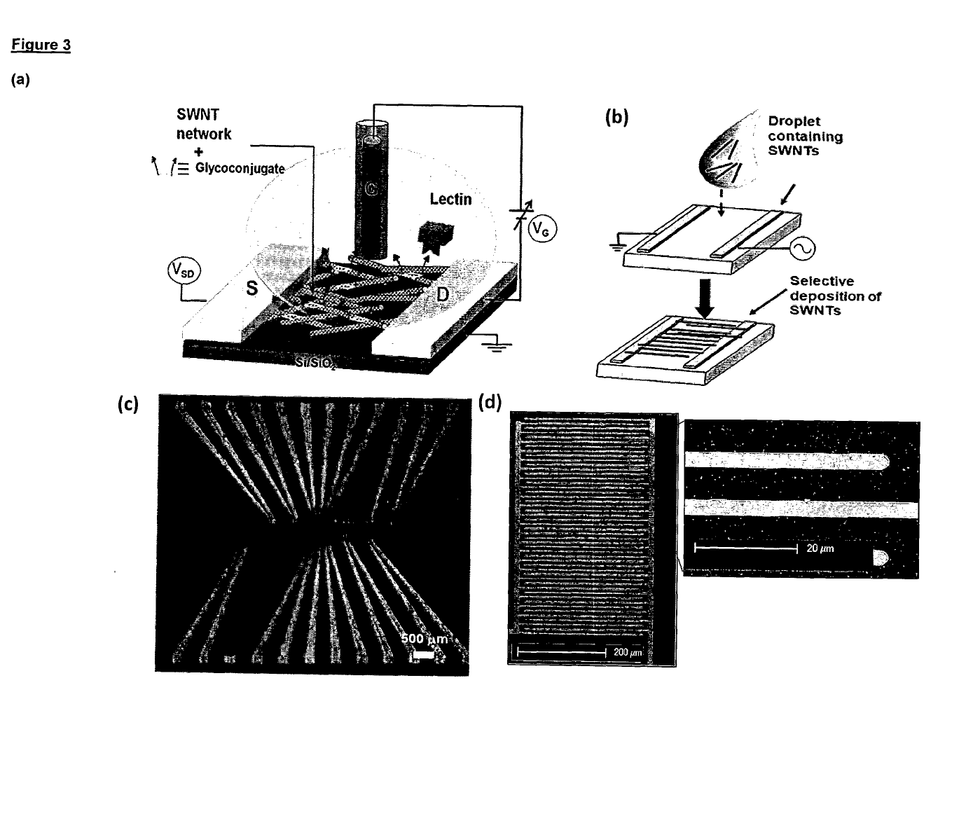

FIG. 3 represents a “SWNT-FET” device (SWNT=“single wall carbon nanotubes” and FET=“Field Effect Transistor”) and its fabrication. More particularly, FIG. 3a is a schematic illustration of glycoconjugate (I) functionalized single walled carbon nanotubes (SWNTs)-FET detection platform for selective detection of lectin. FIG. 3b is a schematic of dielectrophorectic method used for selective deposition of SWNTs onto pre-patterned microelectrodes. FIG. 3c is an optical image of Si/SiO₂ chip with micropatterned interdigitated electrodes. FIG. 3d is a SEM image of interdigitated electrodes used for device fabrication. Inset shows the SWNTs deposited by dielectrophoresis technique between microelectrodes.

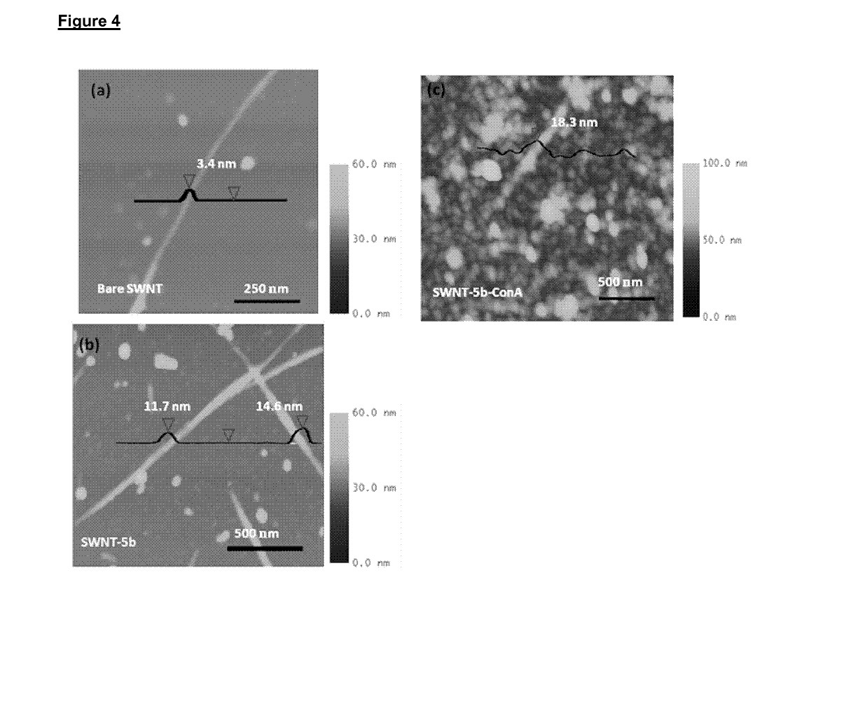

FIG. 4 shows Atomic Force Microscope (AFM) images from bare SWNTs (FIG. 4a), from SWNT functionalized with a glycoconjugate “5b” (defined as “SWNT-5b”) (FIG. 4b) and from this non covalent molecular structure “SWNT-5b” and after ConA lectin attachment (defined as “SWNT-5b-ConA”) (FIG. 4c). Lectin attachment was performed in the presence of 5 μM Ca²⁺.

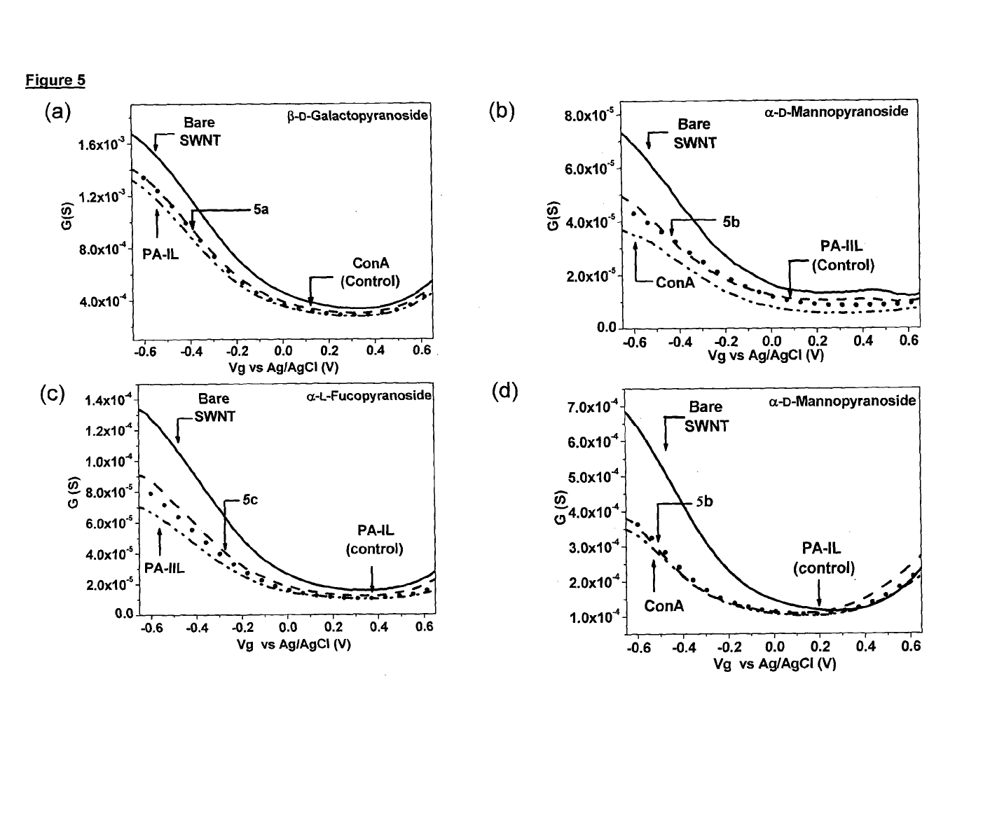

FIG. 5 shows the conductance “G” (which is expressed in siemens (S)) versus gate voltage (“Vg”) of bare SWNT-FET device and after functionalization with respectively the porphyrin based glycoconjugates (I) named “5a” (see FIG. 5a), named “5b” (see FIGS. 5b and 5d) and named “5c” (see FIG. 5c) and after attachment with 5 μM selective lectin and their controls (5 μM). Lectin attachment was performed in the presence of 5 μM Ca²⁺.

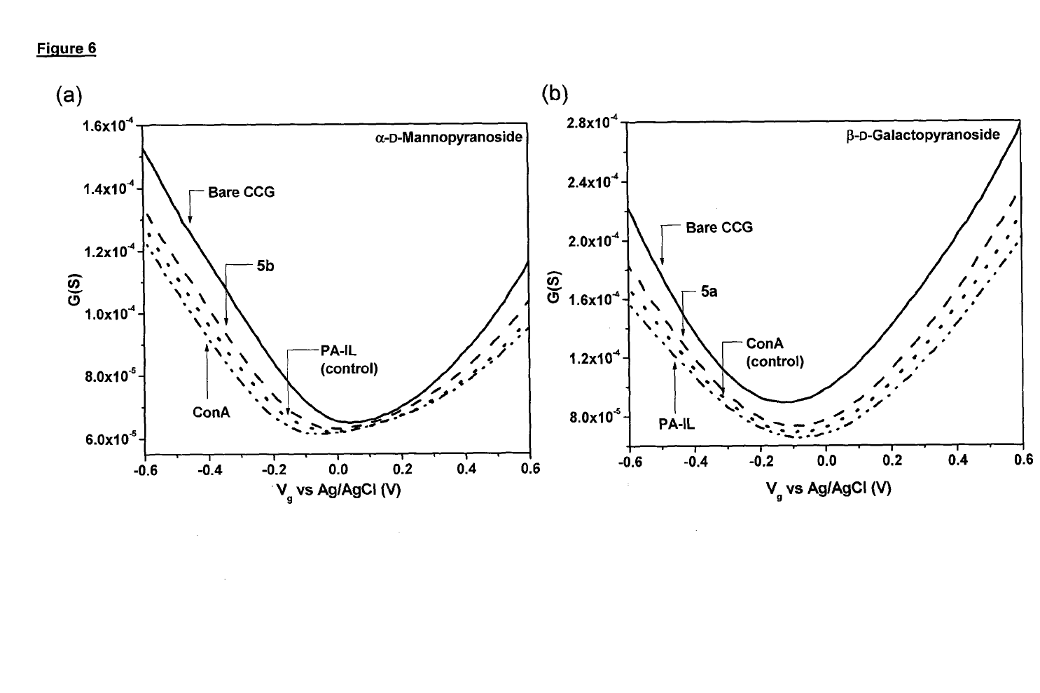

FIG. 6 illustrates the electronic detection of carbohydrate-lectin interactions. More particularly, FIG. 6 shows the conductance “G” (which is expressed in siemens (S)) versus gate voltage (“Vg”) of respectively bare CCG-FET device, and after functionalization with respectively the α-D-mannose porphyrin-based glycoconjugates “5b” (see FIG. 6a) and the β-D-galactose porphyrin-based glycoconjugates “5a” (see FIG. 6b), and after incubation with 2 μM solutions of non-selective lectin (PA-IL) (control) and selective lectin ConA (which is specific for α-D-mannose) and non-selective lectin ConA (control) and selective lectin PA-IL (which is specific for β-D-galactose). The lectin binding was performed in the presence of 5 μM Ca²⁺.

### EXAMPLE I

### Preparation of Three Porphyrin Based Glycoconjugates (I)

The three porphyrin based glycoconjugates prepared here carry respectively β-D-galactose, α-D-mannose and α-L-fucose epitopes.

The general synthesis scheme used in this example for preparing the said porphyrin based glycoconjugates of general formula (I) is illustrated in FIG. 1, wherein a propargyloxy benzaldehyde of general formula (V) leads to a propargyloxy phenyl porphyrin of general formula (IV) which leads to an alkyne-functionalized prophyrin of general formula (III), which in addition to a carbohydrate azido-derivative of general formula (II) leads to the glycoconjugates (I) wherein M=Zn and A is an oxygen atom.

General experimental methods are described for preparing the three following porphyrin based glycoconjugate (1):
5,10,15,20-Tetrakis(4′-{1-[(β-D-galactopyranosyloxy)-3,6-dioxaoct-8-yl]-1,2,3-triazol-4-yl}methyleneoxyphenyl)-Zn-(II)-porphyrin (named “5a” in FIG. 2d),
5,10,15,20-Tetrakis(4′-{1-[(α-D-mannopyranosyloxy)-3,6-dioxaoct-8-yl]-1,2,3-triazol-4-yl}methyleneoxyphenyl)-Zn-(II)-porphyrin (named “5b” in FIG. 2d) and,
5,10,15,20-Tetrakis(4′-{1-[(α-L-fucopyranosyloxy)-3,6-dioxaoct-8-yl]-1,2,3-triazol-4-yl}methyleneoxyphenyl)-Zn-(II)-porphyrin (named “5c” in FIG. 2d).

All reagents were commercial (highest purity available for reagent grade compounds) and used without further purification. Solvents were distilled over CaH₂ (CH₂Cl₂) or Mg/I₂ (MeOH).

Reactions were performed under an argon atmosphere. Reactions under microwave activation were performed on a Biotage Initiator system.

Thin-layer chromatography (TLC) was carried out on aluminum sheets coated with silica gel 60 F₂₅₄ (Merck). TLC plates were inspected by UV light (λ=254 nm) and developed by treatment with a mixture of 10% H₂SO₄ in EtOH/H₂O (95:5 v/v) followed by heating.

Silica gel column chromatography was performed with silica gel Si 60 (40-63 μm).

NMR spectra were recorded at 293 K, unless otherwise stated, using a 300 MHz or a 400 MHz Bruker Spectrometer. Chemical shifts are referenced relative to deuterated solvent residual peaks. The following abbreviations are used to explain the observed multiplicities: s, singlet; d, doublet; t, triplet; q, quadruplet; m, multiplet and bs, broad singlet.

A residual peak at 147.8 ppm was due to the machine and could be usually observed on 75 MHz ¹³C spectra. This residual peak was checked to be independent from the sample analyses. Complete signal assignments were based on 1D and 2D NMR experiments (COSY, HSQC and HMBC). MALDI-ToF mass spectra were recorded in positive ion reflectron mode using a Voyager DE-STR spectrometer (Applied Biosystem).

### 1) General Procedure for 1,3-Dipolar Cycloadditions (Method A)

The alkyne-functionalized porphyrin “2” (of general formula (III)), copper iodide, DIPEA and azido-derivatives “3a” to “3c” (of general formula (II)) in degassed DMF were introduced in a Biotage Initiator 2-5 mL vial. The vial was flushed with argon and protected from light (aluminum sheet) and the solution was sonicated for 30 seconds. The vial was sealed with a septum cap and heated at 110° C. for 10 min under microwave irradiation (solvent absorption level: High). After uncapping the vial, the crude mixture was diluted with EtOAc (200 mL). The organic layer was washed with water (4×50 mL) and brine (50 mL). The organic layer was dried (Na₂SO₄), filtered and evaporated. The crude product was purified by flash silica gel column chromatography to afford the desired acetylated glycoporphyrins “4a” to “4c”.

### 2) General Procedure for Deacetylation (Method B)

The acetylated glycoporphyrins “4a” to “4c” were suspended in distilled MeOH, distilled CH₂Cl₂, ultra-pure water and ultra-pure triethylamine (5:1:1:1, v/v/v/v). The mixture was stirred under argon at room temperature for 3 to 4 days. Solvents were evaporated off then co-evaporated with toluene. The residue was dissolved in ultra-pure water (5 mL) and freeze-dried to afford pure hydroxylated glycoporphyrins “5a” to “5c” (general formula (I)).

The carbohydrate azido-derivatives “3a”,¹ “3b”,² and “3c”³ (general formula (II)), were previously described in the literature and prepared accordingly. The synthesis scheme of these three compounds is respectively illustrated in FIGS. 2a to 2c.

The reagents and conditions used in the steps described in FIG. 2d are given below:

Step a: pyrrole, propionic acid, 120° C.;
Step b: ZnCl₂, microwaves, 120° C.;
Step c: compounds “3a” to “3c”, CuI, iPr₂NEt, DMF, microwaves, 110° C.;
Step d (deacetylation): MeOH, Et₃N, H₂O.

### (a) Preparation of the Compound “3a”¹

### General Formula (II)

### 1-Azido-3,6-dioxaoct-8-yl 2,3,4,6-tetra-O-acetyl-β-D-galactopyranoside

SnCl₄ (8.7 mL, 76.9 mmol, 3 eq.) was added dropwise (within 90 min—syringe pump) at room temperature (r.t) to a stirred solution of 1,2,3,4,6-tetra-O-acetyl-β-D-galactopyranose (10 g, 25.6 mmol), silver trifluoroacetate (8.49 g, 38.4 mmol, 1.5 eq.) and 2-(2-chloroethoxy)ethanol (5.6 mL, 38.4 mmol, 1.5 eq.) in freshly distilled CH₂Cl₂ (400 mL).

The mixture was protected from light. Disappearance of the starting material was observed (TLC monitoring) after 10 minutes following the addition of SnCl₄. The mixture was transferred in saturated aqueous NaHCO₃ (400 mL) and the pH was adjusted above 8. The solution was vigorously stirred for 15 min. The biphasic solution was extracted with CH₂Cl₂ (3×250 mL). The organic layers were combined, washed successively with saturated aqueous NaHCO₃ (2×250 mL), water (2×250 mL) and brine (250 mL) then dried (Na₂SO₄) and filtered. After concentration under high vacuum, contaminants such as metallic salts were removed by filtration on a plug of silica gel (Et₂O/PE, 8:2). Sodium azide (6.3 g, 96.35 mmol) and tetra-n-butyl ammonium iodide (0.712 g, 1.9 mmol) were added to the resulting colorless oil previously dissolved in anhydrous DMF (100 mL). The mixture was stirred at 70° C. under argon for 16 hrs. The mixture was cooled to r.t., transferred into 1 L of brine and extracted with EtOAc (3×250 mL). The organic layers were combined then washed with aq. NaHCO₃ (2×200 mL), water (2×200 mL), brine (200 mL) then dried (Na₂SO₄) and filtered. After concentration under high vacuum, the residue (yellow to orange gum) was purified by silica gel column chromatography (Et₂O/PE, 8:2) to afford the corresponding azido-functionalized β-glycoside “3a” as a colorless gum (8.02 g, 62% over 2 steps).

The ¹H NMR and ¹³C NMR data are given below.

¹H NMR (300 MHz, CDCl₃)

δ 5.37 (dd, J<1 Hz, J=3.4 Hz, 1H, H-4), 5.18 (dd, J=7.9 Hz, J=10.5 Hz, 1H, H-2), 5.00 (dd, J=3.4 Hz, J=10.5 Hz, 1H, H-3), 4.56 (d, J=7.9 Hz, 1H, H-1), 4.07-4.19 (m, 2H, H-6a, H-6b), 3.88-4.01 (m, 2H, OCH₂, H-5), 3.60-3.81 (m, 9H, OCH₂), 3.38 (t, J=5.0 Hz, 2H, CH₂N₃), 1.96, 2.02, 2.04, 2.13 (4s, 4×3H, CH₃CO).

¹³C NMR (100 MHz, CDCl₃)

δ 170.1, 170.0, 169.9, 169.2 (4s, 4×CH₃CO), 101.1 (C-1), 70.6 (C-5), 70.5, 70.4 (2s, 2×CH₂O), 70.4 (C-3), 70.1, 69.8, 68.8, (3s, 3×CH₂O), 68.5 (C-2), 66.8 (C-4), 61.0 (C-6), 50.4 (CH₂N₃), 20.5, 20.4, 20.4, 20.3 (4s, 4×CH₃CO).

### (b) Preparation of the Compound “1”

### General Formula (IV)

### 5,10,15,20-Tetrakis(4′-propargyloxyphenyl)-2H-porphyrin

A solution of p-propargyloxy-benzaldehyde (general formula (V)) (3.6 g, 22.5 mmol, 1 eq.) and pyrrole (1.6 mL, 22.5 mL, 1 eq.) in 5 mL of propionic acid was added dropwise under argon to a pre-heated (120° C.) 500 mL round-bottom flask flushed with argon containing 100 mL of propionic acid. After 1 hour, the mixture was slowly cooled to r.t. over approximately 2 hours. The crude product was precipitated by cooling the mixture with an ice-bath and adding 250 mL of methanol. Filtration afforded a purple gum which was dissolved in CH₂Cl₂. After evaporation of the solvent, the residue was re-dissolved in a minimum amount of CHCl₃ and the dropwise addition of methanol yielded the crystallized porphyrin “1” as pure deep purple compound (1.09 g, 23%).

The ¹H NMR data are given below.

¹H NMR (300 MHz, CDCl₃)

δ 8.87 (s, 8H, H-pyr), 8.14 (d, J=8.4 Hz, 8H, H-ar), 7.36 (d, J=8.4 Hz, 8H, H-ar), 4.98 (d, J=1.9 Hz, 8H, OCH₂C≡CH), 2.70 (t, J=1.9 Hz, 4H, OCH₂C≡CH), 2.76 (s, 2H, NH).

### (c) Preparation of the Compound “2”

### General Formula (III)

### 5,10,15,20-Tetrakis(4′-propargyloxyphenyl)-Zn-(II)-porphyrin (2)⁴

The tetrapropargylated porphyrin “1” (500 mg, 0.60 mmol, 1 eq.) and ZnCl₂ (410 mg, 3.0 mmol, 5 eq.) were introduced into a Biotage Initiator 2-5 mL vial. The vial was flushed with argon and protected from light (aluminum sheet). Anhydrous and degassed DMF (4.5 mL) then Et₃N (585 μL, 4.2 mmol, 7 eq.) were added. The vial was sealed with a septum cap and heated at 120° C. for 15 min under microwave irradiation (solvent absorption level: High). After uncapping the vial, the crude mixture was diluted with EtOAc (250 mL). The organic layer was washed with water (3×100 mL) and brine (100 mL). The organic layer was dried (Na₂SO₄), filtered and evaporated. The crude product was crystallized (CHCl₃/MeOH) to afford the pure zinc-porphyrin “2” as a deep purple solid (434 mg, 87%).

The ¹H NMR data are given below.

¹H NMR (300 MHz, CDCl₃)

δ 8.97 (s, 8H, H-pyr), 8.14 (d, J=8.4 Hz, 8H, H-ar), 7.34 (d, J=8.4 Hz, 8H, H-ar), 4.97 (d, J=1.9 Hz, 8H, OCH₂C≡CH), 2.69 (t, J=1.9 Hz, 4H, OCH₂C≡CH).

### (d) Preparation of the Compound “4a”

### See FIG. 2d

### 5,10,15,20-Tetrakis(4′-{1-[(2,3,4,6-tetra-O-acetyl-β-D-galactopyranosyloxy)-3,6-dioxaoct-8-yl]-1,2,3-triazol-4-yl}methyleneoxyphenyl)-Zn-(II)-porphyrin

Prepared according to method A from compounds “2” (50 mg, 0.056 mmol, 1 eq.), “3a” (169 mg, 0.34 mmol, 6 eq.), copper iodide (5.3 mg, 0.5 eq.) and DIPEA (49 μL, 5 eq.) in DMF (2.5 mL). After work up, the residue was purified by silica gel flash chromatography (EtOAc then EtOAc/MeOH, 95:5) yielding pure compound “4a” as a purple gum (104 mg, 64%).

The ¹H NMR and ¹³C NMR data are given below.

¹H NMR (300 MHz, CDCl₃)

δ 8.92 (s, 8H, H-pyr), 8.11 (d, J=8.5 Hz, 8H, H-ar), 7.72 (s, 4H, H-triazole), 7.25* (d, J=8.5 Hz, 8H, H-ar), 5.34 (dd, J=3.3 Hz, J<1 Hz, 4H, H-4), 5.18 (dd, J=10.5 Hz, J=7.9 Hz, 4H, H-2), 4.97 (dd, J=10.5 Hz, J=3.3 Hz, 4H, H-3), 4.86 (bs, 8H, -PhOCH₂), 4.50 (d, J=7.9 Hz, 4H, H-1), 4.41 (t, J=4.9 Hz, 8H, OCH₂CH₂N), 4.15-4.02 (m, 8H, H-6a, H-6b), 3.98-3.89 (m, 4H, GalOCH₂CH₂O), 3.88-3.83 (m, 4H, H-5), 3.79 (t, J=4.9 Hz, 8H, OCH₂CH₂N), 3.71-3.65 (m, 4H, GalOCH₂CH₂O), 3.64-3.51 (m, 24H, GalOCH₂CH₂OCH₂CH₂O), 2.10, 2.00, 1.95, 1.94 (4s, 4×12H, CH₃CO). *: signal partially overlapped by residual CHCl₃ peak.

¹³C NMR (75 MHz, CDCl₃)

δ 170.5, 170.3, 170.2, 169.6 (4s, CH₃CO), 157.9 (C^{IV}-ar), 150.5 (C^{IV}-pyr), 143.7 (C^{IV}-triazole), 136.4 (C^{IV}-ar), 135.8 (CH-ar), 131.8 (CH-pyr), 123.8 (CH-triazole), 120.4 (Ph-C^{IV}-pyr), 112.9 (CH-ar), 101.4 (C-1), 71.0 (C-3), 70.8 (C-5), 70.8, 70.74, 70.68 (3s, 12C, GalOCH₂CH₂OCH₂CH₂O), 69.4 (OCH₂CH₂N), 69.3 (GalOCH₂—), 68.9 (C-2), 67.1 (C-4), 62.0 (PhOCH₂), 61.3 (C-6), 50.4 (OCH₂CH₂N), 20.9, 20.8, 20.74, 20.70 (4s, CH₃CO).

MALDI-TOF MS: calcd for C₁₃₆H₁₆₀N₁₆O₅₂Zn [M]⁺ 2912.97, found 2912.92.

### (e) Preparation of the Compound “4b”

### See FIG. 2d

### 5,10,15,20-Tetrakis(4′-{1-[(2,3,4,6-tetra-O-acetyl-α-D-mannopyranosyloxy)-3,6-dioxaoct-8-yl]-1,2,3-triazol-4-yl}methyleneoxyphenyl)-Zn-(II)-porphyrin

Prepared according to Method A from compounds “2” (60 mg, 0.067 mmol, 1 eq), “3b” (202 mg, 0.40 mmol, 6 eq.), copper iodide (6.4 mg, 0.5 eq.) and DIPEA (58 μL, 5 eq.) in DMF (3 mL). After work up, the residue was purified by silica gel flash chromatography (EtOAc then EtOAc/MeOH, 95:5) yielding pure compound “4b” as a purple gum (134 mg, 68%).

The ¹H NMR and ¹³C NMR data are given below.

¹H NMR (300 MHz, CDCl₃)

δ 8.92 (s, 8H, H-pyr), 8.10 (d, J=8.5 Hz, 8H, H-ar), 7.66 (s, 4H, H-triazole), 7.22 (d, J=8.5 Hz, 8H, H-ar), 5.38-5.30 (m, 4H, H-3), 5.30-5.24 (m, 4H, H-4), 5.24-5.21 (m, 4H, H-2), 4.85 (d, J=1.4 Hz, 4H, H-1), 4.70 (bs, 8H, PhOCH₂), 4.36 (t, J=4.8 Hz, 8H, OCH₂CH₂N), 4.25 (dd, J=12.1 Hz, J=4.9 Hz, 4H, H-6a), 4.15-3.99 (m, 8H, H-6b, H-5), 3.85-3.70 (m, 12H, OCH₂CH₂N, ManOCH₂CH₂O), 3.69-3.48 (m, 28H, ManOCH₂CH₂O, ManOCH₂CH₂OCH₂CH₂O), 2.10, 2.05, 1.98, 1.95 (4s, 4×12H, CH₃CO).

¹³C NMR (75 MHz, CDCl₃)

δ 170.7, 170.1, 170.0, 169.8 (4s, CH₃CO), 157.8 (C^{IV}-ar), 150.4 (C^{IV}-pyr), 143.5 (C^{IV}-triazole), 136.5 (C^{IV}-ar), 135.8 (CH-ar), 131.7 (CH-pyr), 123.6 (CH-triazole), 120.3 (Ph-C^{IV}-pyr), 112.8 (CH-ar), 97.7 (C-1), 70.7, 70.6, 70.1 (3s, 12C, ManOCH₂CH₂OCH₂CH₂O), 69.6 (C-2), 69.4 (OCH₂CH₂N), 69.1 (C-3), 68.6 (C-5), 67.4 (ManOCH₂—), 66.2 (C-4), 62.5 (C-6), 61.8 (PhOCH₂), 50.4 (OCH₂CH₂N), 21.0, 20.83, 20.79 (3s, 16C, CH₃CO).

MALDI-TOF MS: calcd for C₁₃₆H₁₆₀N₁₆O₅₂Zn [M]⁺ 2912.97, found 2913.10.

### (f) Preparation of the Compound “4c”

### See FIG. 2d

### 5,10,15,20-Tetrakis(4′-{1-[(2,3,4,6-tetra-O-acetyl-α-L-fucopyranosyloxy)-3,6-dioxaoct-8-yl]-1,2,3-triazol-4-yl}methyleneoxyphenyl)-Zn-(II)-porphyrin

Prepared according to method A from compounds “2” (84 mg, 0.094 mmol, 1 eq), “3c” (256 mg, 0.57 mmol, 6 eq.), copper iodide (9.0 mg, 0.5 eq.) and DIPEA (83 μL, 5 eq.) in DMF (3 mL). After work up, the residue was purified by silica gel flash chromatography (EtOAc then EtOAc/MeOH, 90:10) yielding pure compound “4c” as a purple gum (224 mg, 89%).

The ¹H NMR and ¹³C NMR data are given below.

¹H NMR (300 MHz, CDCl₃)

δ 8.91 (s, 8H, H-pyr), 8.10 (d, J=8.5 Hz, 8H, H-ar), 7.67 (s, 4H, H-triazole), 7.22 (d, J=8.5 Hz, 8H, H-ar), 5.38-5.29 (m, 4H, H-3), 5.28-5.22 (m, 4H, H-4), 5.12-5.03 (m, 8H, H-1, H-2), 4.74 (bs, 8H, PhOCH₂), 4.37 (t, J=4.9 Hz, 8H, OCH₂CH₂N), 4.19 (qd, J=6.4 Hz, J<1 Hz, 4H, H-5), 3.79-3.73 (m, 12H, OCH₂CH₂N, FucOCH₂CH₂O), 3.65-3.49 (m, 28H, FucOCH₂CH₂O, FucOCH₂CH₂OCH₂CH₂O), 2.12, 2.00, 1.94 (3s, 3×12H, CH₃CO), 1.10 (d, J=6.5 Hz, 12H, CH₃).

¹³C NMR (75 MHz, CDCl₃)

δ 170.7, 170.5, 170.2 (3s, CH₃CO), 157.9 (C^{IV}-ar), 150.5 (C^{IV}-pyr), 143.8 (C^{IV}-triazole), 136.5 (C^{IV}-ar), 135.8 (CH-ar), 131.7 (CH-pyr), 123.7 (CH-triazole), 120.4 (Ph-C^{IV}-pyr), 112.8 (CH-ar), 96.3 (C-1), 71.3 (C-4), 70.7, 70.3 (2s, 12C, FucOCH₂CH₂OCH₂CH₂O), 69.4 (OCH₂CH₂N), 68.3 (C-2), 68.1 (C-3), 67.4 (FucOCH₂—), 64.5 (C-5), 61.9 (PhOCH₂), 50.4 (OCH₂CH₂N), 20.9, 20.82, 20.76 (3s, 12C, CH₃CO).

MALDI-TOF MS: calcd for C₁₂₈H₁₅₂N₁₆O₄₄Zn [M]⁺ 2680.94, found 2681.01

### (g) Preparation of the Compound “5a”

### General Formula (I)

### 5,10,15,20-Tetrakis(4′-{1-[(β-D-galactopyranosyloxy)-3,6-dioxaoct-8-yl]-1,2,3-triazol-4-yl}methyleneoxyphenyl)-Zn-(II)-porphyrin

Prepared according to method B, compound “4a” (86 mg, 0.029 mmol) was suspended in 5 mL methanol, 1 mL dichloromethane, 1 mL water and 1 mL triethylamine. After stirring at r.t. for 4 days and evaporation of the solvents, the mixture was freeze-dried to afford the pure deacetylated glycoporphyrin “5a” as a freeze-dried purple solid (66 mg, 99%).

The ¹H NMR and ¹³C NMR data are given below.

¹H NMR (400 MHz, DMSO-d₆+εD₂O)

δ 8.81 (s, 8H, H-pyr), 8.39 (s, 4H, H-triazole), 8.09 (d, J=8.5 Hz, 8H, H-ar), 7.47 (d, J=8.5 Hz, 8H, H-ar), 5.44 (bs, 8H, PhOCH₂), 4.64 (t, J=5.1 Hz, 8H, OCH₂CH₂N), 4.12 (d, J=7.2 Hz, 4H, H-1), 3.92 (t, J=5.1 Hz, 8H, OCH₂CH₂N), 3.89-3.80 (m, 4H, H-6a), 3.64-3.46 (m, 40H, H-5, H-6b, GalOCH₂CH₂OCH₂CH₂O), 3.38-3.23 (m, 12H, H-2, H-3, H-4).

¹³C NMR (100 MHz, DMSO-d₆+εD₂O)

δ 157.8 (C^{IV}-ar), 149.7 (C^{IV}-pyr), 142.8 (C^{IV}-triazole), 135.4 (C^{IV}-ar), 135.3 (CH-ar), 131.7 (CH-pyr), 125.4 (CH-triazole), 120.0 (Ph-C^{IV}-pyr), 113.0 (CH-ar), 103.66 (C-1), 75.2, 73.4, 70.5 (3s, C-2, C-3, C-4), 69.9, 69.8, 69.7 (3s, 12C, GalOCH₂CH₂OCH₂CH₂O), 68.9 (OCH₂CH₂N), 68.1 (C-5), 67.9 (C-6), 61.5 (PhOCH₂), 60.5 (GalOCH₂—), 49.7 (OCH₂CH₂N).

MALDI-TOF MS: calcd for C₁₀₄H₁₂₈N₁₆O₃₆Zn [M]⁺ 2240.80, found 2240.78

### (h) Preparation of the Compound “51D”

### General Formula (I)

### 5,10,15,20-Tetrakis(4′-{1-[(α-D-mannopyranosyloxy)-3,6-dioxaoct-8-yl]-1,2,3-triazol-4-yl}methyleneoxyphenyl)-Zn-(II)-porphyrin

Prepared according to method B, compound “4b” (118 mg, 0.040 mmol) was suspended in 5 mL methanol, 1 mL dichloromethane, 1 mL water and 1 mL triethylamine. After stirring at r.t. for 4 days and evaporation of the solvents, the mixture was freeze-dried to afford the pure deacetylated glycoporphyrin “5b” as a freeze-dried purple solid (80 mg, 88%).

The ¹H NMR and ¹³C NMR data are given below.

¹H NMR (400 MHz, DMSO-d₆+εD₂)

δ 8.81 (s, 8H, H-pyr), 8.39 (s, 4H, H-triazole), 8.09 (d, J=8.5 Hz, 8H, H-ar), 7.46 (d, J=8.5 Hz, 8H, H-ar), 5.44 (s, 8H, PhOCH₂), 4.70-4.59 (m, 12H, H-1, OCH₂CH₂N), 3.92 (t, J=5.1 Hz, 8H, OCH₂CH₂N), 3.73-3.52 (m, 40H, H-2, H-6a, H-6b, ManOCH₂CH₂O, ManOCH₂CH₂OCH₂CH₂O), 3.50-3.29 (m, 16H, H-3, H-4, H-5, ManOCH₂CH₂O).

¹³C NMR (100 MHz, DMSO-d₆+εD₂O) δ 157.8 (C^{IV}-ar), 149.7 (C^{IV}-pyr), 142.8 (C^{IV}-triazole), 135.5 (C^{IV}-ar), 135.4 (CH-ar), 131.7 (CH-pyr), 125.3 (CH-triazole), 120.0 (Ph-C^{IV}-porph), 113.0 (CH-ar), 100.0 (C-1), 74.0, 70.9 (2s, C-3, C-4 or C-5), 70.3 (C-2), 69.8, 69.74, 69.66 (3s, 12C, ManOCH₂CH₂OCH₂CH₂O), 68.9 (OCH₂CH₂N), 66.9 (C-4 or C-5), 65.8 (C-6), 61.5 (PhOCH₂), 61.3 (ManOCH₂—), 49.7 (OCH₂CH₂N).

MALDI-TOF MS: calcd for C₁₀₄H₁₂₈N₁₆O₃₆Zn [M]⁺ 2240.80, found 2240.84

### (i) Preparation of the Compound “5c”

### General Formula (I)

### 5,10,15,20-Tetrakis(4′-{1-[(α-L-fucopyranosyloxy)-3,6-dioxaoct-8-yl]-1,2,3-triazol-4-yl}methyleneoxyphenyl)-Zn-(II)-porphyrin

Prepared according to method B, compound “4c” (202 mg, 0.075 mmol) was suspended in 5 mL methanol, 1 mL dichloromethane, 1 mL water and 1 mL triethylamine. After stirring at r.t. for 4 days and evaporation of the solvents, the mixture was freeze-dried to afford the pure deacetylated glycoporphyrin “5c” as a freeze-dried purple solid (155 mg, 94%).

The ¹H NMR and ¹³C NMR data are given below.

¹H NMR (400 MHz, DMSO-d₆+εD₂O)

δ 8.80 (s, 8H, H-pyr), 8.37 (s, 4H, H-triazole), 8.07 (d, J=8.4 Hz, 8H, H-ar), 7.44 (d, J=8.4 Hz, 8H, H-ar), 5.41 (s, 8H, PhOCH₂), 4.68-4.57 (m, 12H, H-1, OCH₂CH₂N), 3.90 (t, J=5.1 Hz, 8H, OCH₂CH₂N), 3.81 (q*, J=6.3 Hz, 4H, H-5), 3.68-3.43 (m, 44H, H-2, H-3, H-4, FucOCH₂CH₂OCH₂CH₂O), 1.06 (d, J=6.5 Hz, 12H, CH₃). * The coupling constant between H-5 and H-4 was too small to be observed.

¹³C NMR (100 MHz, DMSO-d₆+εD₂O)

δ 157.9 (C^{IV}-ar), 149.8 (C^{IV}-pyr), 142.9 (C^{IV}-triazole), 135.5 (C^{IV}-ar), 135.4 (CH-ar), 131.7 (CH-pyr), 125.4 (CH-triazole), 120.1 (Ph-C^{IV}-porph), 113.0 (CH-ar), 99.4 (C-1), 71.7 (C-4), 70.0, 69.84, 69.82 (3s, 12C, FucOCH₂CH₂OCH₂CH₂O), 69.7 (C-3), 69.0 (OCH₂CH₂N), 68.1 (C-2), 66.9 (FucOCH₂—), 66.1 (C-5), 61.5 (PhOCH₂), 49.8 (OCH₂CH₂N), 16.7 (CH₃).

MALDI-TOF MS: calcd for C₁₀₄H₁₂₈N₁₆O₃₂Zn [M]⁺ 2176.82, found 2176.90.

### EXAMPLE II

### Fabrication of an Electronic Nano-Detection Device “SWNT-FET” and its Use for the Detection of Lectins

### 1) Fabrication of an Electronic Nano-Detection Device Named “SWNT-FET” Device

In this example, the used carbon nanostructures are carbon nanotubes and more particularly single-walled carbon nanotubes (SWNTs).

Single-walled carbon nanotubes (SWNTs) were procured from Carbon Solutions Inc. and were used as conducting channels in the Field-Effect Transistor (FET) devices described below.

Field-effect transistor (FET) devices were fabricated by patterning interdigitated microelectrodes (source-drain spacing of 5 μm) on top of 200 nm oxide layer on silicon substrates using photolithography and e-beam evaporation of 30 nm titanium and 100 nm of gold (FIGS. 3c and 3d).

Alternating current dielectrophoresis (a.c DEP) technique was used for selective deposition of SWNT networks from DMF (N,N-dimethylformamide) suspension onto each interdigitated microelectrodes pattern (FIG. 3b).

Each silicon chip (2 mm×2 mm) comprising of multiple FET devices was then placed onto a standard ceramic dual in-line package (CERDIP) and wirebonded.

Two Keithley 2400 sourcemeters were used for FET measurements.

The electrical performance of each such obtained “SWNT-FET” device was investigated in electrolyte gated FET device configuration. The conductance of SWNT-FET device was tuned using the electrolyte as a highly effective gate. A small fluid (1 mL) chamber was placed over the SWNT-FET device to control the liquid environment using phosphate buffer solution (PBS) at pH 7. A liquid gate potential (−0.75V to 0.75 V) with respect to the grounded drain electrode was applied using Ag/AgCl (3 M KCl) reference electrode submerged in the electrolyte. The drain current of the device was measured at a constant source-drain voltage of 50 mV.

2) Non Covalent Functionalization of SWNT-FET with Glycoconjugates (I)

To selectively detect lectins, the SWNT-FET device surface thus obtained is non covalently functionalized with respectively the three porphyrin based glycoconjugates (I) such as prepared in example I.

The Sugar (or carbohydrate) which is present at the extremity of each of these glycoconjugates (I) is respectively the β-D-galactosyl (for glycoconjugate “5a”), the α-D-mannosyl (for “5b”) and the α-L-fucosyl (for “5c”).

Here is thus investigated the specific interactions between three different sugars, namely β-D-galactose, α-D-mannose and α-L-fucose with respectively the three following lectins: PA-IL, ConA, and PA-IIL, by using the above mentioned non covalently functionalized SWNT-FET device (see FIG. 3a).

PA-IL is a bacterial lectin isolated from Pseudomonas aeruginosa that is specific for β-D-galactose and expressed in recombinant form in Escherichia coli.

PA-IIL is a bacterial lectin isolated from Pseudomonas aeruginosa that is specific for α-L-fucose and expressed in recombinant form in Escherichia coli.

These lectins PA-IL and PA-IIL were produced by the Inventors.⁸

ConA (25 kDa) is a plant lectin from Canavalia ensiformis that is specific for α-D-mannose and is available commercially from Sigma and used without further purification.

Surface functionalization of SWNT FET device with each porphyrin based glycoconjugate respectively named “5a”, “5b” and “5c” was performed by incubating them in their 5 μM solution in deionized water for 2 hours followed by rinsing with deionized water. This step was followed by incubating the chips for 30 minutes in different concentrations of lectin solutions prepared in PBS with 5 μM CaCl₂ and latter rinsing with PBS solution.

Imaging studies: The scanning electron microscopy (SEM) was performed with a Phillips XL30 FEG at acceleration voltage of 10 keV (FIG. 3d).

Atomic force microscope (AFM) images (FIG. 4) were obtained using scanning probe microscope (Veeco Nanoscope II) in a tapping mode configuration. Samples were prepared by spin coating of bare or functionalized SWNTs onto a freshly cleaved sheet of mica. The images were taken after 30 min of drying in ambient and subsequent washing with PBS solution (for functionalized SWNTs).

FIG. 4a depicts a small bundle of bare SWNTs with diameter of 3.4 nm. After non covalent functionalization with glycoconjugate “5b” (non covalent molecular structure “SWNT-5b”), SWNT bundles show diameters of 11.7-14.6 nm (FIG. 4b). Con A lectin binding to the functionalized “SWNT-5b” nanostructures (“SWNT-5b-ConA”) increases SWNT diameters to 18.3 nm (FIG. 4c). The AFM results indicate specific binding of Con A lectin to α-D-mannose glycoconjugate “5b” on the surface of SWNTs.

### 3) Results and Discussion

The electronic detection of the interactions between the sugar (carbohydrate) of the glycoconjugates (I) and lectin molecules is illustrated by the curves of the FIG. 5.

FIG. 5 shows the conductance G vs V_{g} curves for SWNT-FET at different stages of glycoconjugate-lectin interactions.

In FIGS. 5b and 5d, the bare SWNT exhibited initially a p-type behavior which upon functionalization with α-D-mannose glycoconjugate “5b” resulted in shift of the threshold voltage to negative values and a decrease in conductance.

Later when SWNT-FET device was treated with PA-IIL lectin (1 μM) (a control lectin for α-D-mannose), no significant change in G vs Vg curve was observed (FIG. 5b). The similar result was observed with another control PA-IL lectin (FIG. 5d).

However on treating with ConA lectin (specific binding to α-D-mannose) a negative shift in threshold voltage and further decrease in conductance was observed.

This shift and decrease in conductance indicates a positive interaction between Con A lectin and α-D-mannose glycoconjugate “5b”.

Both the proteins i.e. control (PA-IIL (Ip=3.9)) and Con A (Ip=5) have isoelectric points (Ip)<7, implying that they possess a net negative charge at measured conditions (pH=7).

Hence upon attachment positive binding of specific lectin on SWNT FET (p-type) there is a shift in threshold voltage and decrease in overall conductance.

Conversely, non covalent functionalization with β-D-galactose glycoconjugate “5a” results in selective response to galactophilic lectin PA-IL and not to Con A as indicated in FIG. 5a.

In FIG. 5c, a decrease in conductance is observed for experiment in presence of PA-IIL lectin compared to PA-IL lectin (which is a control lectin with no affinity for fucose). This decrease in conductance in addition to a shift in the threshold voltage indicates positive interactions between the PA-IIL lectin and the fucosylated glycoconjugate “5c”.

### EXAMPLE III

### Fabrication of an Electronic Nano-Detection Device “CCG-FET” and its Use for the Detection of Lectins

1) Fabrication of an electronic nano-detection device named “CCG-FET” device

In this example, the carbon nanostructure used is graphene or specifically chemically converted graphene (CCG). More particularly, there is prepared here as previously described in the literature⁵⁻⁷ chemically reduced graphene oxide, which is also known in the literature as chemically converted graphene (CCG).

Briefly, graphite oxide was synthesized utilizing a modified Hummers' method on graphite flakes (Sigma Aldrich) that underwent a preoxidation step.⁶ Graphite oxide (˜0.125 wt %) was exfoliated to form graphene oxide via 30 minutes of ultrasonification followed by 30 minutes of centrifugation at 3400 revolutions per minute (r.p.m.) to remove unexfoliated graphite oxide (GO). Graphene oxide was then reduced to RGO with hydrazine hydrate (Sigma Aldrich) following the reported procedure^{5,7}, the chemically converted graphene (CCG) thus obtained being then used as conducting channels in the FETs.

As described in example II, metal interdigitated devices (Au/Ti, 100 nm/30 nm) with interelectrode spacing of 10 μm were patterned on a Si/SiO₂ substrate using conventional photolithography.

Each chip (2 mm×2 mm in size) containing four identical FET devices was then set into a 40-pin (CERDIP) and wirebonded using Au wire.

Devices were subsequently isolated from the rest of the package by epoxying the inner cavity. CCG were deposited onto each interdigitated microelectrodes pattern by a.c. DEP method from a suspension in DMF (Agilent 33250A 80 MHz Function/Arbitrary Waveform Generator, a.c. frequency (10 MHz), bias voltage (8 Vₚₚ), bias duration (60 s))⁹ in order to obtain the “CCG-FET” device.

“RGO-FET” devices were prepared using the same a.c. DEP technique but with different parameters (a.c. frequency (300 kHz), bias voltage (10.00 Vₚₚ), bias duration (120 s)).¹⁰

The electrical performance of each such obtained “CCG-FET” device was investigated in electrolyte gated FET device configuration as described in example II.

2) Non Covalent Functionalization of CCG-FET with Glycoconjugates (I)

To selectively detect lectins, the CCG-FET device surface thus obtained is non covalently functionalized with respectively the α-D-mannose porphyrin based glycoconjugates “5b” and the β-D-galactose porphyrin based glycoconjugates “5a”.

The specific interactions between—the α-D-mannose (“5b”) and the ConA lectin (FIG. 6a) and—the β-D-galactose (“5a”) and the PA-IL lectin (FIG. 6b) are investigated by using the non covalently functionalized CCG-FET device.

Surface functionalization of the CCG-FET device with porphyrin based glycoconjugate “5b” or “5a” was performed by incubating the chips in 20 μM of the glycoconjugates solution (in deionized water) for 2 hours followed by rinsing three times with double-distilled water. After testing the transfer characteristics, the chips were incubated for 40 min in different concentrations of lectin solutions prepared in PBS with 5 μM CaCl₂ and subsequently washed three times with PBS solution. For each glycoconjugate functionalized device, non-specific lectins were tested first, followed by washing procedures and measuring of specific lectin. The final transfer characteristics were tested again in the configuration mentioned above.

### 3) Results and Discussion

The electronic detection of the interactions between the sugar (carbohydrate) of the glycoconjugates (I) and lectin molecules is illustrated by the curves of the FIG. 6.

More particularly FIG. 6 shows the curves (conductance “G” versus gate voltage (V_{g})) for CCG-FET at different stages of glycoconjugate-lectin interactions.

In FIGS. 6a and 6b, the bare CCG exhibited initially a p-type behavior which upon functionalization with α-D-mannose glycoconjugate “5b” (FIG. 6a) or with β-D-galactose glycoconjugate “5a” (FIG. 6b) resulted in shift of the threshold voltage to negative values and a decrease in conductance.

When CCG-FET device thus functionalized with α-D-mannose glycoconjugate “5b” was treated with PA-IL lectin (2 μM) (a control lectin for α-D-mannose), no significant change in G vs Vg curve was observed (FIG. 6a).

The similar result was observed in FIG. 6b: when CCG-FET device thus functionalized with β-D-galactose glycoconjugate “5a” was treated with conA lectin (2 μM) (also a control lectin for β-D-galactose), no significant change in G vs Vg curve was observed.

However on treating the CCG-FET device functionalized with α-D-mannose glycoconjugate “5b” with ConA lectin (specific binding to α-D-mannose) a negative shift in threshold voltage and further decrease in conductance was observed (FIG. 6a).

This shift and decrease in conductance indicates a positive interaction between Con A lectin and α-D-mannose glycoconjugate “5b”.

Both the proteins i.e. control (PA-IIL (Ip=3.9)) and Con A (Ip=5) have isoelectric points (Ip)<7, implying that they possess a net negative charge at measured conditions (pH=7).

Hence upon attachment positive binding of specific lectin on CCG-FET (p-type) there is a shift in threshold voltage and decrease in overall conductance.

On treating the CCG-FET device functionalized with β-D-galactose glycoconjugate “5a” with PA-IL lectin (specific binding to β-D-galactose) a negative shift in threshold voltage and further decrease in conductance was observed (FIG. 6b).

### REFERENCES

(1) Szurmai, Z.; Szabó, L.; Lipták, A. Acta Chim. Hung. 1989, 126, 259-269.
(2) Li, J.; Zacharek, S.; Chen, X.; Wang, J.; Zhang, W.; Janczuk, A.; Wang, P. G. Bioorg. Med. Chem. 1999, 7, 1549-1558.
(3) Sanki, A. K.; Mahal, L. K. Synlett 2006, 455-459.
(4) Olson, M. A.; Coskun, A; Klajn, R.; Fang, L.; Dey, S. K.; Browne, K. P.; Grzybowski, B. A.; Stoddart, J. F. Nano Lett. 2009, 9, 3185-3190.
(5) Li, D. et al. Nature Nano 2008, 3, 101-105.
(6) Kovtyukhova, N. I. et al. Chem. Mater. 1999, 11, 771-778.
(7) Kotchey, G. P. et al., Enzymatic oxidation of graphene oxide. ACS Nano 2011, 5, 2098-2108.
(8) a) Blanchard, B. et al. J. Mol. Biol. 2008, 383, 837-853. (b) Mitchell, E. P. et al. Proteins: Struct. Funct. Bioinfo. 2005, 58, 735-746.
(9) Vedala H. et al. Nano Lett. 2011, 11, 170-175.
(10) Vijayaraghavan, A. et al. ACS Nano 2009, 3, 1729-1734.

1. Non covalent molecular structure characterized in that it comprises a carbon nanostructure and a porphyrin based glycoconjugate (I) which is linked to the said carbon nanostructure by a non covalent link,
the said glycoconjugate (I) having the formula: 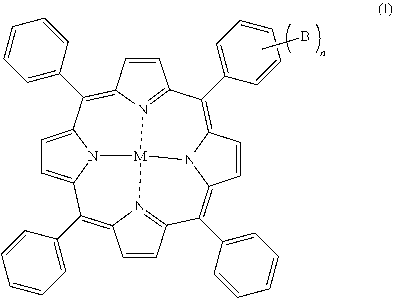
wherein
M is a metal selected in the group comprising Fe, Ni, Zn, Cu, Mn, Cr or Co,
B is a group which is present on at least one of the four phenyl group (C₆H₅) represented in (I),
n is an integer from 1 to 3, that is to say that one to three B group(s) may be present on each phenyl group,
and B is represented by a -A-C group
wherein
A is selected in the group comprising an oxygen atom (O), a sulfur atom (S), a NH group
or a (CH₂)ₙ₁ group, n₁ being an integer from 1 to 10,
C is a group of formula: 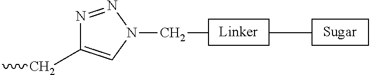
wherein
the linker is a group of formula: 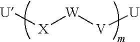
wherein
m is an integer from 0 to 15
U′, U=absent or is CH₂ with the proviso that when m=0 then
if one of U′ or U is absent then the other is CH₂,
X=CH₂, O, CO (carbonyl)
W=CH₂, NH
V=CH₂, C₆H₄ (phenyl “Ph”)
the sugar is a group having at least one carbohydrate moiety and is selecting in the group comprising: 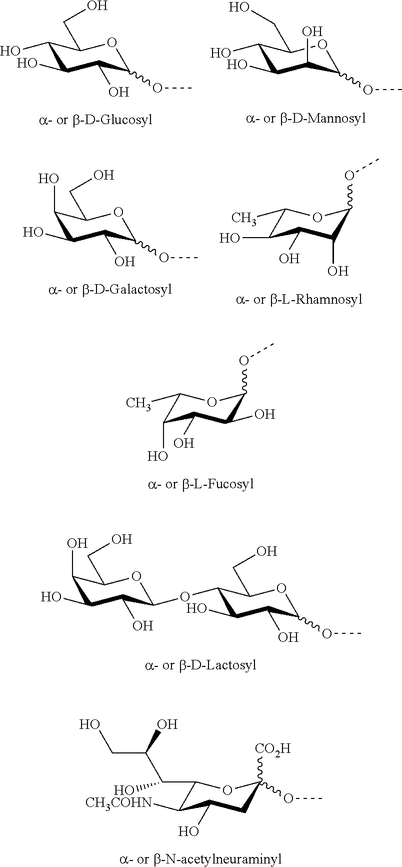
and their derivatives.

2. Non covalent molecular structure according to claim 1, wherein the sugar derivatives in the C group are selected in the group comprising:

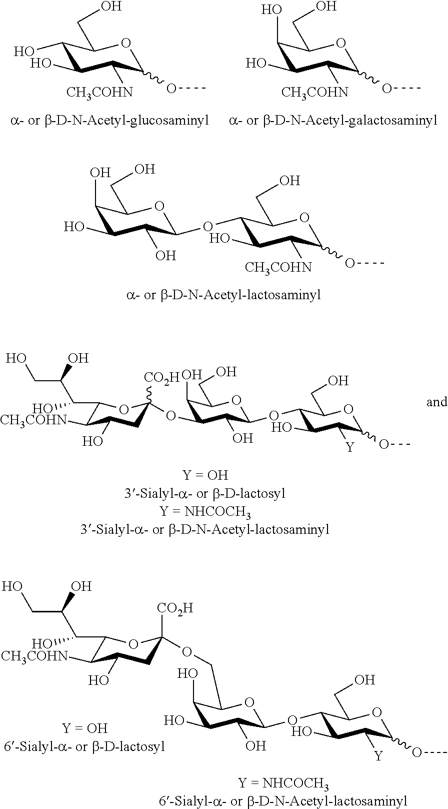

3. Non covalent molecular structure according to claim 1, wherein the sugar derivatives in the C group are selected in the group comprising:

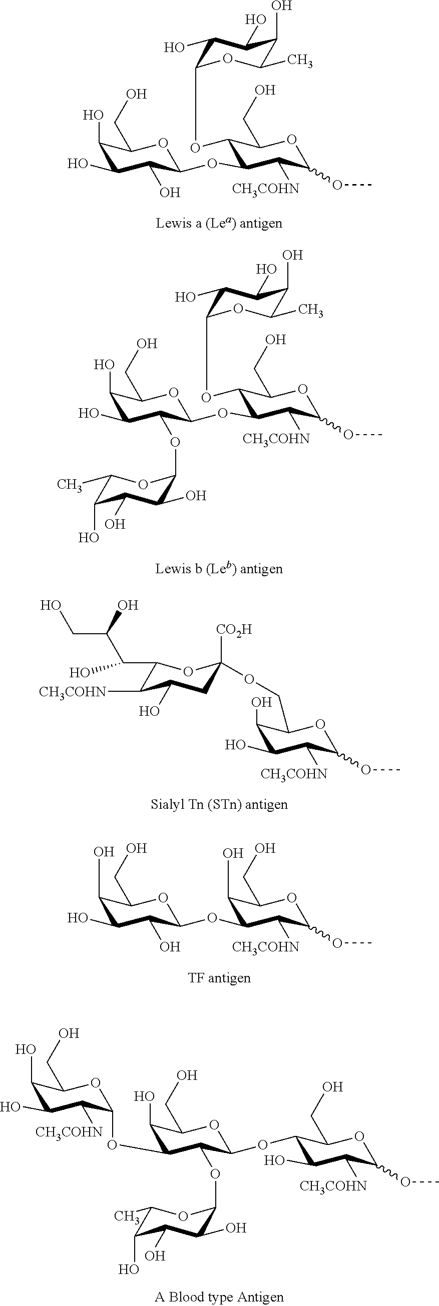

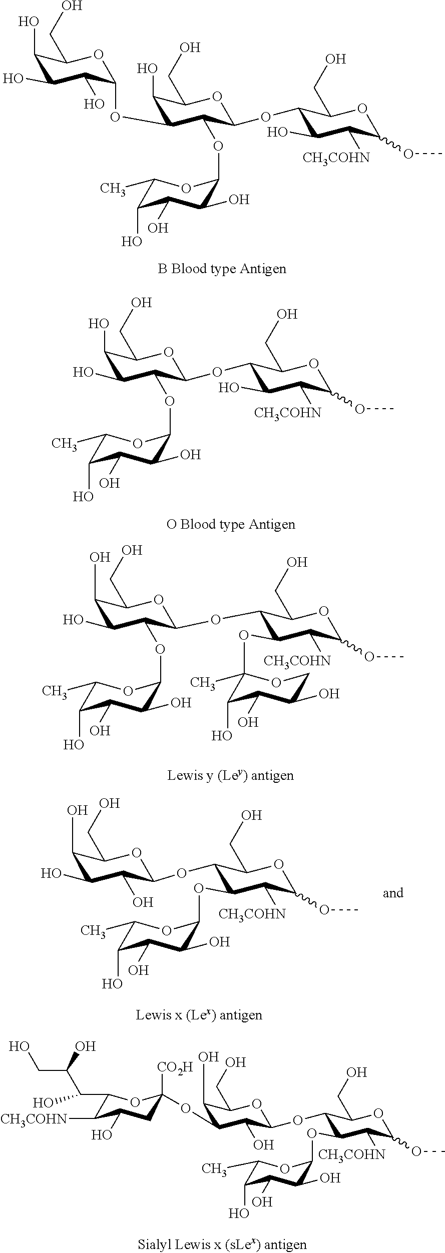

4. Non covalent molecular structure according to: claim 1, wherein the linker defined in the C group is selected in the group comprising:
m=0, U′=absent and U=CH₂,
m=0, U′=U=CH₂,
m=1, U′=U=absent, X=W=V=CH₂,
m=2, U′=U=absent, X=W=V=CH₂,
m=1, U′=CH₂, U=absent, X=O, W=V=CH₂,
m=2, U′=CH₂, U=absent, X=O, W=V=CH₂,
m=2, U′=absent, U=V=CH₂, X=CO, W=NH and
m=1, U′=U=absent, X=CO, W=NH and V=Ph.

5. Non covalent molecular structure according to claim 1, wherein the B group is present on each of the four phenyl group and when:
n=1, B is in the para-position of each phenyl group,
n=2, the two B are in the two meta-position of each phenyl group,
n=3, the three B are in the para-position and in the two meta-position of each phenyl group.

6. Non covalent molecular structure according to claim 1, wherein in the porphyrin based glycoconjugate (I), A is an oxygen group, n=1 or 2 and M is Zn, the said glycoconjugate (I) being selected in the group comprising:

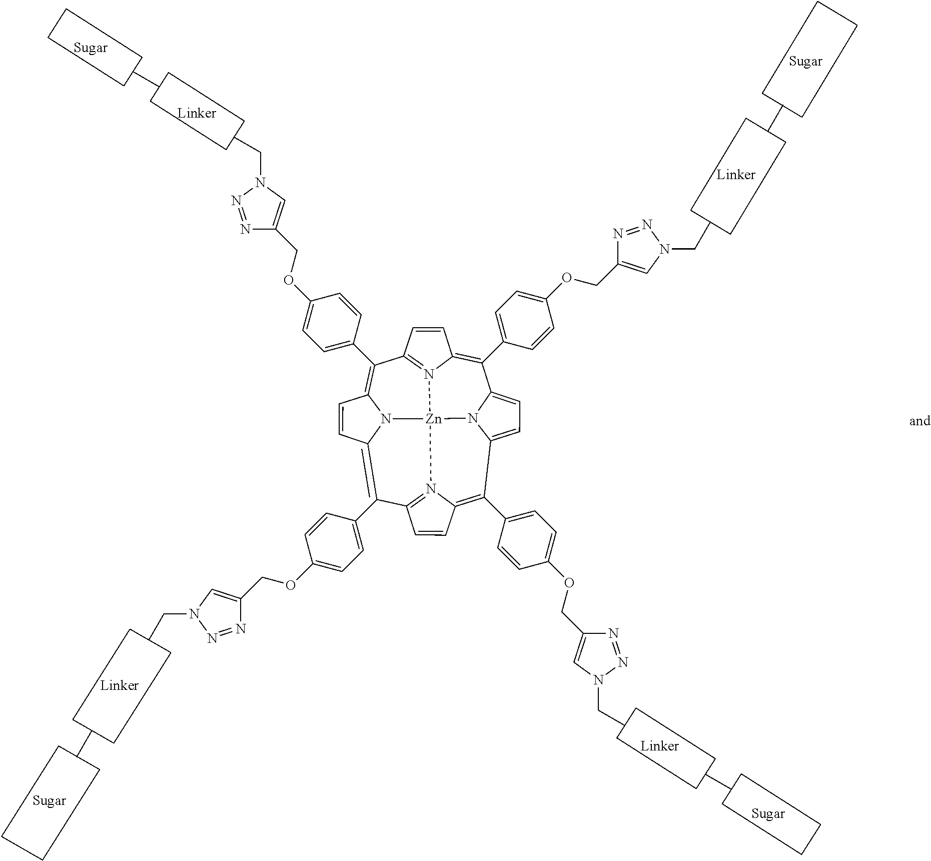

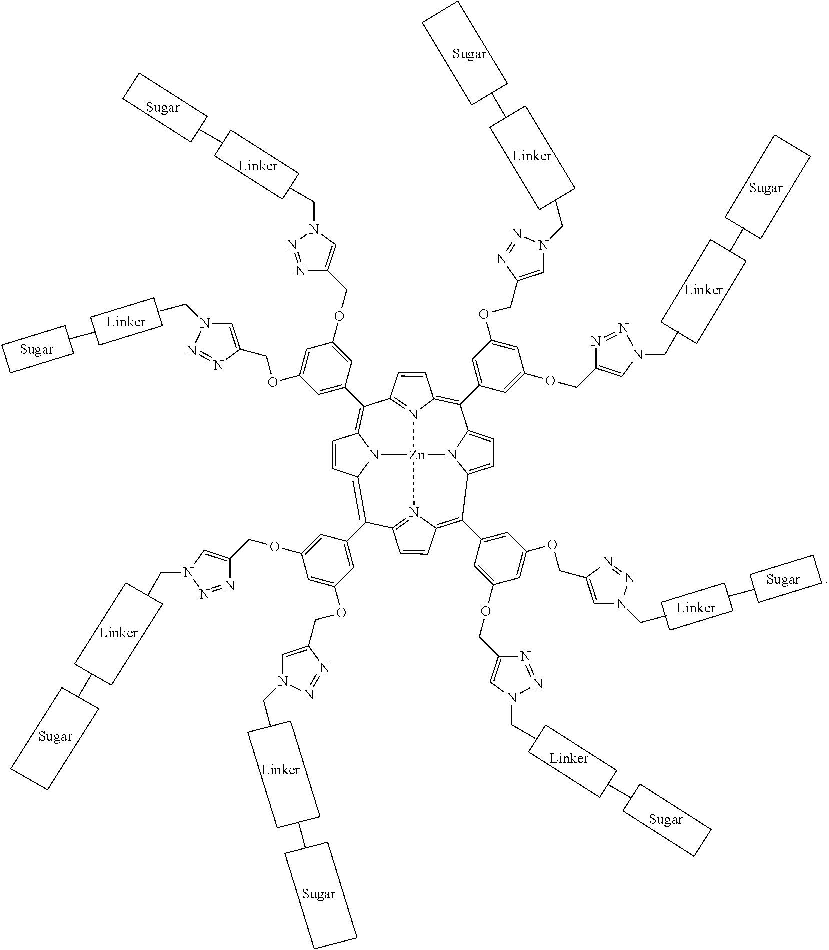

7. Non covalent molecular structure according to claim 6, wherein in the porphyrin based glycoconjugate (I):
the linker is CH₂—(O—CH₂—CH₂)₂,
the sugar is selected in the group comprising β-D-galactosyl, α-D-mannosyl and α-L-fucosyl.

8. Non covalent molecular structure according to claim 1, wherein the carbon nanostructures are selected in the group comprising carbon nanotubes, graphene, graphitic onions, cones, nanohorns, nanohelices, nanobarrels and fullerenes.

9. Non covalent molecular structure according to claim 8, wherein the carbon nanostructures are graphene and carbon nanotubes, the said carbon nanotubes being selected in the group comprising Single Wall Carbon Nanotubes (SWCNTs), Double Wall Carbon Nanotubes (DWCNTs), Triple Wall Carbon Nanotubes (TWCNTs) and Multi Wall Carbon Nanotubes (MWCNTs).

10. Non covalent molecular structure according to claim 1, wherein the non-covalent link between the carbon nanostructures and the glycoconjugate (I) is a π-π type interaction.

11. A device for detecting a lectin characterized in that it comprises a non covalent molecular structure according to claim 1.

12. A device according to claim 11 which is an electronic nano-detection device and which comprises a field effect transistor (FET),
the said device comprising:
carbon nanostructures bridging two metal electrodes respectively called “source” (S) and “drain” (D),
a third electrode called “gate” (G) connected either to a substrate layer or to an electrode immersed in a solution covering the said device (“liquid gate”).

13. A device according to claim 12 wherein the two metal electrodes (S) and (D) are spacing each other from 1 nm to 10 cm, preferably from 1 cm to 2.5 cm and more preferably from 1 μm to 10 μm.

14. A device according to claim 12, wherein the substrate layer is an insulator.

15. Method for detecting the presence of a lectin in a sample to be analysed characterized in that it comprises the following steps:
using a device according to claim 11,
bringing the lectin to be analysed in contact with the non covalent molecular structure,
detecting a molecular interaction between the lectin and the sugar of the porphyrin based glycoconjugate (I) of the said non covalent molecular structure, said molecular interaction being detected by a change of the conductive properties of the carbon nanostructures resulting in a change of the electric signal of the said device.

16. Method according to claim 15, wherein the lectin is selected in the group comprising Pseudomonas aeruginosa first lectin (PA-IL), Pseudomonas aeruginosa second lectin (PA-IIL), Concanavalin A (Con A) lectin, Burkholderia cenocepacia A (Bc2L-A) lectin, Burkholderia cenocepacia B (Bc2L-B) lectin, Burkholderia cenocepacia C (Bc2L-C) lectin, Burkholderia ambifaria (Bamb541) lectin, Ralstonia solanacearum (RSL) lectin, Ralstonia solanacearum second lectin (RS-IIL) and Chromobacterium violaceum (CV-IIL) lectin.

17. Method according to claim 15, wherein the preparation of the device comprises the following steps:
forming two metal electrodes (S) and (D) on the substrate layer connected to (G),
adding, between the two electrodes (S) and (D), the carbon nanostructures and then a porphyrin based glycoconjugate (I) in order to form a non covalent molecular structure.

18. Method according to claim 15, wherein the preparation of the device comprises the following steps:
forming two metal electrodes (S) and (D) on the substrate layer connected to (G),
adding, between the two electrodes (S) and (D), a non covalent molecular structure.

19. Method according to claim 15, wherein the preparation of the device comprises the following steps:
generating carbon nanostructures on the substrate layer connected to (G) (by a chemical vapour deposition (CVD) process),
forming two metal electrodes (S) and (D) around the carbon nanostructures,
adding a porphyrin based glycoconjugate (I) in order to form a non covalent molecular structure.
